(19) United States
(12) Patent Application Publication
(10) Pub. No.: US 2007/0020644 A1
(43) Pub. Date: Jan. 25, 2007

(54) Method for detection and characterization of short nucleic acids

(76) Inventors: Alexander A. Kolykhalov, Mountain View, CA (US); A. Rosanna Schroeder, San Mateo, CA (US)

Correspondence Address: PATTON BOGGS LLP, 8484 WESTPARK DRIVE, SUITE 900, MCLEAN, VA, 22102, US

(21) Appl. No.: 11/340,830

(22) Filed: Jan. 26, 2006

Related U.S. Application Data
(60) Provisional application No. 60/647,317, filed on Jan. 26, 2005.

(51) Int. Cl. C12Q 1/68; C12P 19/34

(52) U.S. Cl. 435/6; 435/91.2

(57) ABSTRACT

A method of detecting and characterizing a target short RNA is provided.

### CROSS-REFERENCE TO RELATED APPLICATIONS

This application claims benefit of U.S. provisional patent application Ser. No. 60/647,317, filed Jan. 26, 2005, which is herein incorporated by reference.

### BACKGROUND OF THE INVENTION

Utilization of double-stranded RNA to inhibit gene expression in a sequence-specific manner has revolutionized the drug discovery industry. In mammals, RNA interference (RNAi), is mediated by 17- to 49-nucleotide long, RNA molecules referred to as small interfering RNAs (siRNAi) and microRNAs (mRNA). Short RNA can be synthesized chemically or enzymatically outside of cells and subsequently delivered to cells (see, e.g., Fire, et al., Nature, 391:806-11 (1998); Tuschl, et al., Genes and Dev., 13:3191-97 (1999); and Elbashir, et al., Nature, 411:494-498 (2001)); or can be expressed in vivo by an appropriate vector in cells (see, e.g., U.S. Pat. No. 6,573,099). In addition to their impact on gene expression, these short RNAs may find utility in areas of therapeutics and drug discovery, e.g. as drug targets or as pharmaceutical agents. Thus, in some circumstances, it may be important to know precisely how much of each short RNA exists in cells. In some cases, it may further be important to compare levels of short RNA in different tissue types or before and after application of a stimulus, e.g. a chemical or physical intervention. In addition, many short RNAs go through additional enzymatic or chemical steps as part of the pathway to generate an active RNA agent in vivo. Thus, in order to characterize the different RNAs in a particular synthesis pathway it may be important to characterize the nucleotide sequence at the 3′ end of such short RNAs.

Because many short RNAs such as small interfering RNAs (siRNAs) and micro RNAs (mRNAs) may be present in low amounts in cells, it is desirable that methods of detection be both sensitive and specific. Therefore it is important that methods of detection distinguish between populations of short RNAs by utilizing as much of the sequence information in the target short RNA as possible. Also it is becoming increasingly clear that short RNAs on the order of 15-50 nucleotides can play an important role in gene expression and are difficult to quantify and characterize by methods presently known in the art. It is well established in the art that amplification of a target sequence from a complex systems, such as genomic DNA or total cellular RNA, using only two specific primers often results in multiple “wrong” sequences amplified to the extent that the target sequence can not be visualized among all other sequences. To overcome this problem, a second round of amplification is performed with a second pair of specific primers, the method known as a “nested PCR”. Alternatively, all nucleic acids amplified after the first round of PCR are transferred to a membrane and the target is visualized by hybridization to a probe that is homologous to an internal sequence of the target. The last idea is utilized in TaqMan™ technique: two target specific primers are used to amplify the target and an internal TaqMan probe is used to visualize the correct sequence among numerous “parasitic” amplified sequences. The major feature of short RNAs that makes them completely different from any “regular” RNA is that they are too short to perform any of the standard techniques of amplification and detection. Thus there is a need for an improved method for the detection, quantification and characterization of short RNA species.

To date, the principal methods used for quantification of short RNAs are based on gel electrophoresis (see WO 04/057017 to Dahlberg, James, E., et. al.). Short RNAs are detected either by Northern blotting or by the presence of radioactive RNase-resistant duplexes. Northern blotting and chip hybridization methods have relatively low analytical sensitivity (Krichevsky et al. RNA 9, 1274-1281 2003), so microgram quantities of RNA are needed for analyses; moreover, transfer of short RNAs to filters can introduce problems with quantification of reproducibility of and not typically amenable to high-throughput methods. Moreover, detection methods based on RNase resistance require highly radioactive probes. Further, assays based solely on probe hybridization may not provide adequate discrimination between isotypes closely related in sequence. Alternative approaches involve cloning the target short RNAs and then sequencing the inserts. While this approach may be suitable for discriminating single-base differences between closely related mRNA species, it is time consuming, laborious and also not amenable to high-throughput protocols.

In addition to quantifying and characterizing the RNA populations in a cell, it is also of interest to characterize the effect of processing by various RNA processing enzymes. For example, small interfering RNAs (siRNAs) are short RNA molecules involved in cell defense, such against viral RNA, via a response termed RNA interference (RNAi) (Cullen, B. R., Nature Immunology, 3: 597-599 (2002). One class of siRNAs is produced through the action of the Dicer enzyme and RNA-induced silencing complex (RISC) protein complex. It is of interest to know the nature of the 3′ end of the small RNA after processing by Dicer or other RNA processing enzymes. What are needed are efficient and accurate methods of detecting, quantifying and characterizing target short RNAs for example mRNA and siRNA.

Current methods that utilize reverse transcription and PCR to amplify target short RNA for subsequent detection often utilize a nucleic acid primer specific for the RNA in question. Normally, the length of the complementary part of the primers for reverse transcription is 13-18 nucleotides or longer. Decreasing this length results in dramatically decreased specificity of reverse transcription. For this reason, use of shorter primers, such as random hexamers in low temperature reverse transcription is always followed by a high temperature amplification step utilizing two specific primers, each 15-30 nucleotides long. Short RNAs, such as siRNAs or mRNAs, have total length often only in 18-25 nucleotides range, that makes the hexamer strategy inapplicable for short RNA detection. Incorporation of specially modified nucleotides in RT primers (such as LNA modification or the “minor groove binders”) or designing primers such that the formed primer:RNA double-stranded helix is extended by an adjacent helix part of the primer itself (the “looped” primers, such as one shown on FIG. 2) allows to use shorter targeting primers (having only 6-8 complimentary nucleotides) for more specific reverse transcription at higher temperatures. It can also “extend” the generated cDNA in order to have enough length for PCR amplification, and even for TaqMan detection. What is needed in the art is a method of amplification and quantitative detection which allows for the utilization of the entire small RNA sequence during detection and which improves the signal to noise ratio associated with these assays.

### SUMMARY OF THE INVENTION

The present invention relates to compositions and methods for the detection and characterization of target short nucleic acids, such as small interfering RNAs and other short nucleic acid molecules. More particularly, the present invention relates to improved methods for the quantitative detection of short RNAs containing fewer than 22-25 nucleotides in which a great deal of specificity can be achieved in detection.

In one embodiment the present invention provides a method comprising hybridizing a target short RNA and at least one ligation agent that comprises a nucleic acid that contains a portion of sequence that is not complementary to the target short RNA and a portion of sequence that is complementary to the target short RNA to generate a bound complex, and using a template-dependent ligase enzyme to form a ligated molecule. In one embodiment of the invention, an appropriate substrate for ligase is formed. For example, in circumstances where complementarity exists between the ligation agent and the target short RNA, a ligation substrate will form and a resulting ligated molecule is detected. In other aspects, the appropriate substrate for ligation is not formed when, for example complementarity does not exist between the ligation agent and the target short RNA, or gaps or overhangs occur between the ligation agent and the target short RNA and potential sequences of the target short RNA are not ligated. In some embodiments of this invention, the ligated molecule can be detected with greater specificity than by known methods for detecting short RNAs. In some aspects, the target short RNA is an mRNA, while in yet other aspects, the target short RNA is an siRNA, processed RNA derived from shRNA, or any short RNA molecule 15-30 nucleotides long.

In some embodiments, the ligated molecule is detected and quantified by means of a quantitative nucleic acid amplification technique. For example, in some embodiments, the ligated molecule is mixed with a set of primers and a TaqMan™ probe under conditions appropriate for real time PCR. In other embodiments the ligated molecule is larger than the starting small RNA and can be detected by any method known in the art, including, but not limited to, sequencing assays, polymerase chain reaction assays, hybridization assays, hybridization assays employing a probe complementary to a mutation, microarray assays, bead array assays, primer extension assays, enzyme mismatch cleavage assays, branched hybridization assays, NASBA assays, molecular beacon assays, cycling probe assays, ligase chain reaction assays, invasive cleavage structure assays, ARMS assays, and sandwich hybridization assays. In some preferred embodiments, the detecting step is carried out using a cell lysate.

In one aspect of the invention, short RNA can be distinguished from a sample containing more than one sequence of RNA with greater specificity than methods which do not include an initial ligation step. In another aspect of the invention, the ligation of a ligation agent to short RNA from a sample containing more than one sequence of RNA can be used to distinguish between specifically bound short RNA and non-specifically bound RNA. In yet another aspect of this invention, additional detection specificity is conferred over methods using a non-ligated bound complex by probing with one or more nucleic acid probes that is specific for the sequence of the nucleic acid derived from mostly the target short RNA sequence rather than from mostly the primer sequence.

In one embodiment of the invention, the ligation agent used to form the bound complex comprises a nucleic acid template with one or more sites with sufficient complementarity to the short RNA so as to allow the RNA to hybridize to the template and form a substrate for a template-dependent ligation enzyme. In some aspects, the ligation agent used to form the bound complex with the short RNA comprises a template with six or fewer sites complementary to the short RNA. In some aspects, the method comprises detection of a ligated molecule by a quantitative nucleic acid amplification technique.

### DESCRIPTION OF THE DRAWINGS

FIG. 1 is a simplified flow chart describing one embodiment of a method of the present invention.

FIG. 2 is a simplified flow chart describing one of the methods known in the art that allow higher temperature specific reverse transcription of RNA utilizing long primers with short complimentary part. When short complimentary part of a specially designed primer anneals to a cognate target RNA sequence, the double-stranded part is efficiently extended into the primer. This effect significantly increases stability of the annealed complex, and allows efficient extension of the primers' 3′end into a cDNA.

FIG. 3 shows the result of siRNA detection as an example of short RNA detection utilizing the primer ligation method of detection.

FIG. 4 is a graphic representation showing how the method described in FIG. 1 distinguishes between specific and non-specific target sequences.

FIG. 5 shows the results of detection of the same siRNA molecule with the specific and a mismatched ligation primer.

### DETAILED DESCRIPTION

To facilitate an understanding of the present invention, a number of terms and phrases are defined below: As used herein, the term “siRNA” refers generally to small interfering RNA. As used herein, the term “siRNA target sequence” refers generally to the small interfering RNA desired to be detected (e.g., in the presence of other nucleic acids). As used herein, the term “RNAi” refers generally to interfering RNA. There is no particular limitation in the length of the short RNA molecules that can be characterized and quantitated by the method of this invention. Short RNAs can be, for example, 17 to 49 nucleotides in length, preferably 17 to 35 nucleotides in length, and are more preferably 17 to 29 nucleotides in length. The short RNAs may contain double-stranded RNA portions where such portions are completely homologous, contain non-paired portions due to sequence mismatch (the corresponding nucleotides on each strand are not complementary) or the short RNAs may contain a bulge (lack of a corresponding complementary nucleotide on one strand), and the like.

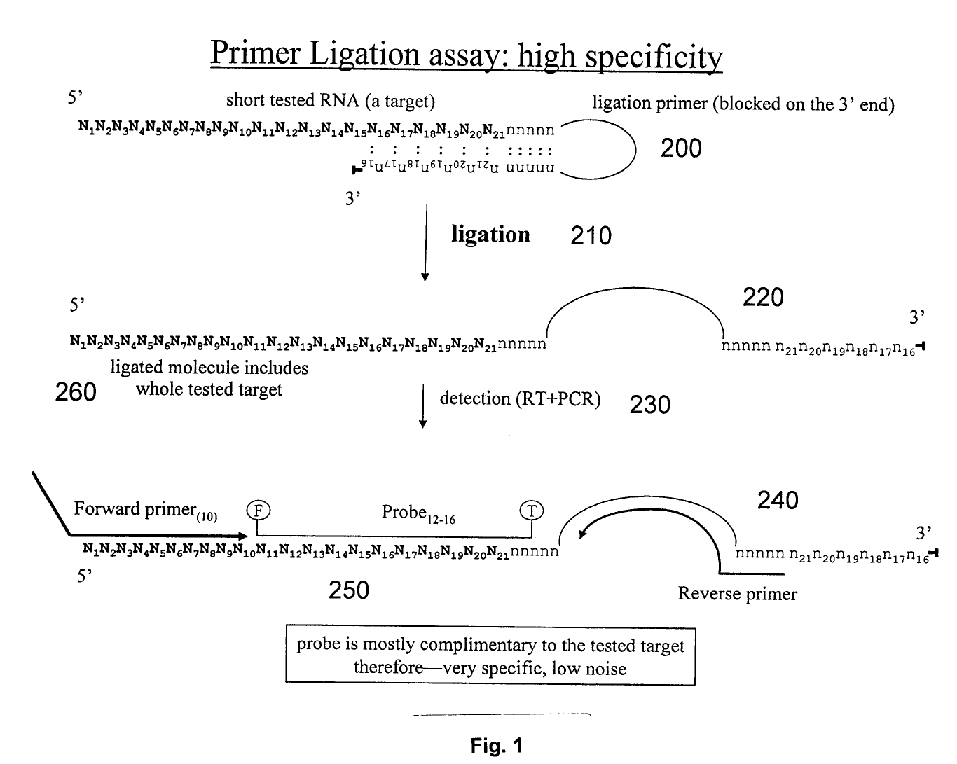

As used herein, the term “RNA bound complex” (see in FIG. 1 at 200) refers to a structure formed by hybridizing a ligation agent with a target short RNA, e.g., an mRNA, shRNA or siRNA. As used herein the term ligation agent refers generally to a nucleic acid for example an oligonucleotide. The ligation agent can be for example a nucleic acid larger than the target short RNA with a small region of homology to the target short RNA. In other embodiments, the ligation agent comprises a hairpin structure that hybridize to the target short RNA to form a bigger hairpin. In a preferred embodiment, a ligation agent has “blocked” 3′ end, that can not be extended with a reverse transcriptase or other polymerases. In preferred embodiments, a ligation agent and the short RNA form a bound complex which is a substrate for a template-specific ligase.

As used herein, the term ligated molecule (220) refers to a structure formed by ligating the ligation agent (e.g., an oligonucleotide) to a target short RNA, e.g., mRNA or siRNA. In preferred embodiments, ligated molecules are capable of being detected using known nucleic acid detection methods, including, but not limited to, those as disclosed herein.

The term homology and homologous refers generally to a degree of identity between nucleotide segments. There may be partial homology or complete homology. A partially homologous sequence is less than 100% identical to another sequence.

A template-dependent ligase refers generally to a class of enzymes (for example DNA ligase) that catalyze phosphodiester bond formation between nucleotides where the nucleotides are positioned adjacent on a nucleic acid template.

The term hybridization refers generally to the pairing of complementary nucleic acids. Hybridization and the strength of hybridization (i.e., the strength of the association between the nucleic acids) is influenced by such factors as the degree of complementary between the nucleic acids, stringency of the conditions involved, and the melting temperature (Tₘ) of the formed hybrid. Hybridization methods involve the annealing of one nucleic acid to another, complementary nucleic acid.

The complement of a nucleic acid sequence as used herein refers generally to an oligonucleotide which, when aligned with the nucleic acid sequence such that the 5′ end of one sequence is base paired with the 3′ end of the other, is in “antiparallel association.” Certain bases not commonly found in naturally occurring nucleic acids may be included in the nucleic acids of the present invention and include, for example, locked nucleic acid (LNA), inosine and 7-deazaguanine.

Complementarity need not be perfect; stable duplexes may contain mismatched base pairs or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs.

The term oligonucleotide is used generally to describe a polymeric molecule comprising two or more deoxyribonucleotides or ribonucleotides, preferably at least 4 nucleotides long, more preferably at least about 10-15 or about 15 to 60 nucleotides. The exact size will depend on many factors, which in turn depend on the ultimate function or use of the oligonucleotide. An oligonucleotide may be generated in any manner known in the art.

When two different, non-overlapping oligonucleotides anneal to different regions of the same linear complementary nucleic acid sequence, and the 3′ end of a first oligonucleotide points towards the 5′ end of a second oligonucleotide, the first oligonucleotide may be called the “upstream” oligonucleotide and the second oligonucleotide may be called the “downstream” oligonucleotide.

The term substantially single-stranded when used in reference to a nucleic acid substrate means generally that the nucleic acid substrate exists primarily as a single strand of nucleic acid in contrast to a double-stranded substrate which exists as two strands of nucleic acid which are held together by inter-strand base pairing interactions.

The term template refers generally to a strand of nucleic acid on which a complementary copy is built from nucleoside triphosphates through the activity of a template-dependent nucleic acid polymerase. Within a duplex the template strand is, by convention, depicted graphically as the “bottom” strand. Similarly, the non-template strand is often depicted graphically as the “top” strand.

The phrase quantitative nucleic acid amplification technique generally refers to a technique which involves monitoring the progress of the nucleic acid amplifications using a feedback means to measure the amount of amplification product for example, by use of an oligonucleotide probe having a fluorescent reporter molecule at one end and a quencher molecule at the other end. The quencher molecule substantially quenches any fluorescence from the reporter molecule when the oligonucleotide probe is intact, and the reporter is substantially unquenched whenever the oligonucleotide is digested by the 3′ exonuclease activity of the polymerase that is copying the template strand. This type of probe is sometimes referred to as a “TaqMan” probe. Quantitative PCR by this technique is described in U.S. Pat. No. 5,538,848 which issued on Jul. 23, 1996 to Livak et al., the disclosure of which is incorporated herein by reference. Related probes and quantitative amplification procedures are described further in U.S. Pat. No. 5,716,784, which issued on Feb. 10, 1998 to Di Cesare et al. and U.S. Pat. No. 5,723,591, which issued on Mar. 3, 1998 to Livak et al., the disclosures of which are incorporated herein by reference. Instruments for carrying out quantitative PCR in microtiter plates are available from Applied Biosystems, 850 Lincoln Centre Drive, Foster City, Calif. 94404 under the trademark ABI Prism® 7700.

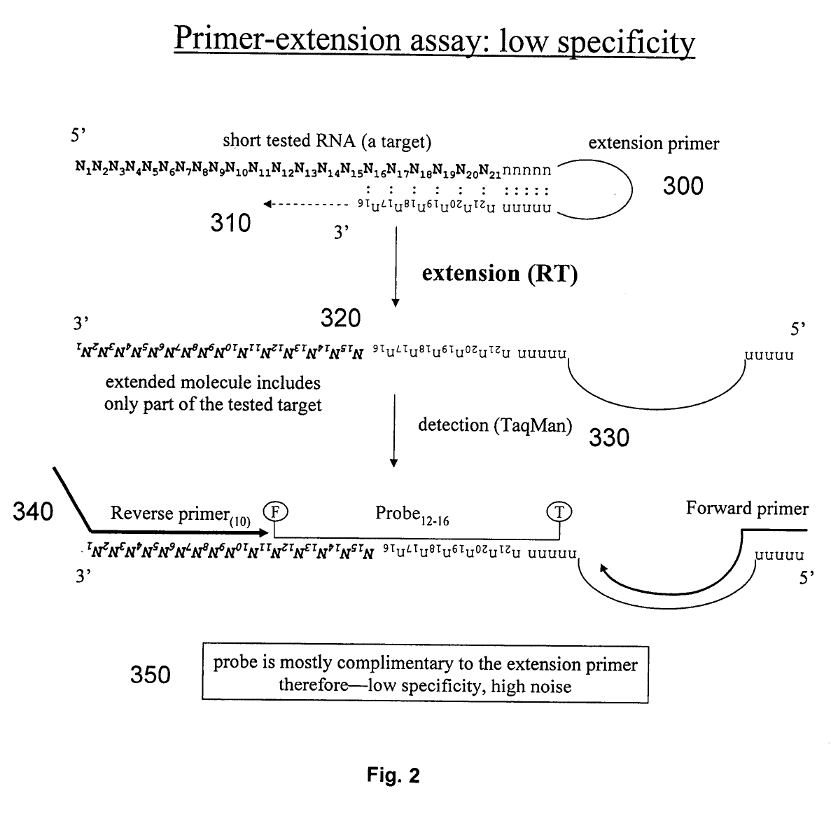

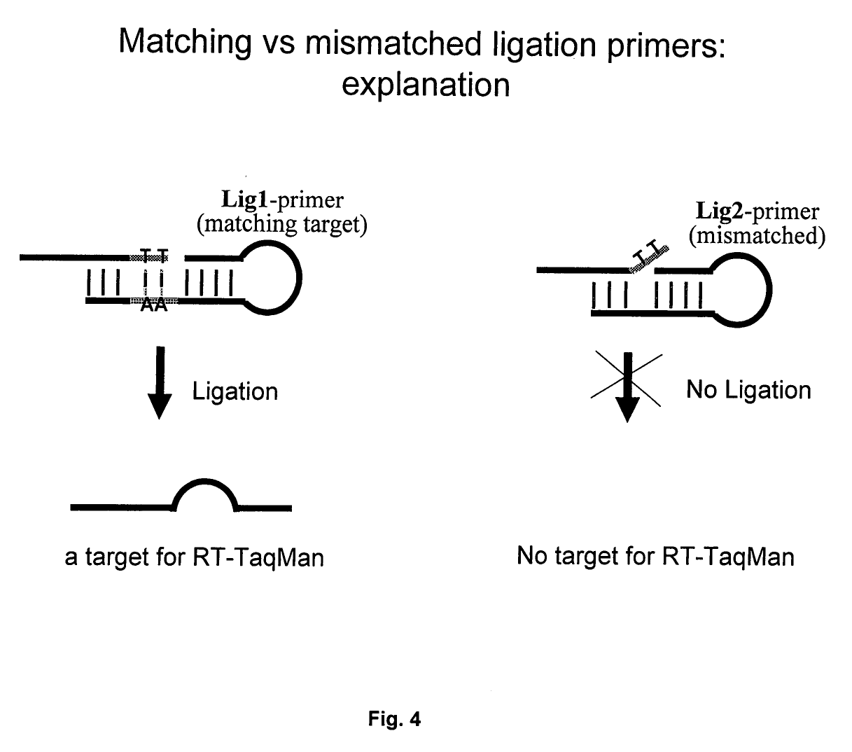

The present invention relates to compositions and methods for the detection and characterization of short RNAs e.g. RNAi agents such siRNAs, mRNAs or RNAs produced by processing of shRNAs. The present invention provides improved methods for detecting, characterizing and quantifying expression of short RNAs. FIGS. 2 and 4 depict a simplified schematic of an embodiment of the present invention and how such methods can enhance the distinction between specific target short RNA and non-specific RNAs that may be present in a system. A system refers generally to a cell, cell lysate, homogenated tissue, or organism. In some embodiments, the present invention provides methods for detecting the expressed and processed short RNA, comprising adding a ligation agent to a population of processed RNAs to form a bound complex. The bound complex (shown in FIG. 2 at 200) that results is then ligated with a template-dependent ligation enzyme, as shown in step 210. The resulting species is a ligated molecule (shown in FIG. 2 at 220) that is then detected using any suitable method including, but not limited to, sequencing assays, polymerase chain reaction assays, hybridization assays, hybridization assays employing a probe, complementary to a mutation, microarray assays, bead array assays, primer extension assays, enzyme mismatch cleavage assays, branched hybridization assays, NASBA assays, molecular beacon assays, cycling probe assays, ligase chain reaction assays, invasive cleavage structure assays, ARMS assays, and sandwich hybridization assays. In some preferred embodiments, the detecting step is carried out using a cell lysate. While the following description focuses on the characterization, detection and quantification of short RNAs such as RNAi agents, it should be understood that the invention also finds use with other short nucleic acid molecules (e.g., DNA and RNA of less than, for example, 40, 30, 20 or 15 nucleotides in length).

In some embodiments of this invention, the ligation agent is a nucleic acid. In particular embodiments, the ligation agent is a DNA oligonucleotide. In a more particular embodiment, the 3′ end of the ligation agent is not a substrate for a chain extending enzyme, for example reverse transcriptase or other polymerases. In some aspects of this invention the 3′ end of ligation agent is blocked by a blocking group comprising, but not limited to hydrogen, 3′-phosphoglycolate or 3′ amine.

In some embodiments of this invention, the ligated molecule that is the resulting product of the ligation of the ligation agent and the target short RNA is further modified following the ligation step. In particular embodiments the ligated molecule is further processed by a quantitative reverse transcription followed by nucleic acid amplification technique, as shown in step 230 of FIG. 2. In other embodiments of this invention, the ligated molecule can be detected by any other means known in the art. In a particular embodiment, the ligated molecule can be detected by means of a Taqman assay using a Taqman probe and specific primers, as shown in step 240. In a preferred embodiment of this aspect of the invention, the Taqman probe is mostly or exclusively complementary to the targeted short RNA (as shown in FIG. 2 at 250). In another embodiment of this invention, the ligated molecule can be detected with a nucleic acid probe specific to any region of the target short RNA.

In some embodiments of this invention the ligation agent does not form an appropriate substrate for a template dependent ligase due to, for example, sequence mismatching overhangs or gaps. In other aspects of this invention the bound complex will form a substrate for a template dependent ligase, the ligated molecule then serves as a substrate for a chain extending enzyme such as, but not limited to reverse transcriptase. In some embodiments the chain extension reaction serves as an assay for the sequence of the target short RNA. In another embodiment of this invention the 3′ end of the short RNA is characterized by probing with a series of ligation agents. In this embodiment of the invention only the ligation agent that forms a duplex region with the target short RNA without gaps or overhangs can act as a substrate for a template-dependent ligase and is consequently detected. In this embodiment of the invention, the nature of the 3′ end of a target short RNA is determined.

In some embodiments of this invention, the target short RNA can be quantified by using a quantitative nucleic acid amplification technique to detect the ligated molecule in a sequence specific manner. In some aspects of this embodiment of this invention, an oligonucleotide probe, for example a TaqMan probe, will be used as part of a sequence-specific, quantitative nucleic acid amplification detection assay. A preferred aspect of this invention is that most of the sequence derived from the target short RNA will be available for detection when it is part of the ligated molecule. Oligonucleotide probes can be designed to be complementary to most or all of the target short RNA to be detected. FIG. 2 shows schematically that only ligated molecules formed from the target short RNA to be detected will contain essentially the complete sequence of the short RNA (260) while RNA bound non-specifically to the nucleic acid will not incorporate any sequence specific to the target short RNA being detected. It is this aspect of the invention which enables maximum sequence information to be utilized when discriminating between target short RNA and other RNAs that may populate a system (260).

FIG. 1 shows how the present invention differs from current methods known in the art, whereby a bound complex is extended and sequence information that was specific to the target short RNA becomes incorporated into both specifically bound and non-specifically bound complex (320) as part of the assay. More specifically, a short RNA and an extending agent form a bound complex 300. A primer extension is performed on the bound complex 300, as shown in step 310, and an extended nucleic acid molecule 320 is formed. The extended nucleic acid molecule is further processed by a quantitative nucleic acid amplification technique, as shown in step 330 of FIG. 2. The extended nucleic acid can be processed by a Taqman assay using Taqman probe, and specific primers, as shown in step 340. The major drawback of this method is that it reduces the effective part necessary for specific amplification and detection by the length of the RT-primer complimentary part. Since the “specific” part of the primers is very short in this case, they will be also incorporated in many wrong cDNAs. This fact renders the part of the target sequence, brought-in with a primer, as not useful for “specific” amplification and detection. That leaves only 12-14 nucleotides from the original typical 20 nucleotide target for specific amplification and detection, that is not enough in typical experiments. As shown in step 350, since the probe is largely complimentary to a sequence derived from a primer rather than from the target, both targeted short RNAs and non-specific RNAs are scored as “detected”, that often results in high noise background. In other words, the TaqMan probe that can be used to quantify the RNA will have reduced specificity and the assay will contain a lower signal to noise ratio compared to the methods of this present invention. In one embodiment of the present invention, the ligated molecule resulting from the ligation of the ligation agent and the target short RNA can be specifically detected and quantified by methods known in the art (for example as mentioned previously with a TaqMan assay and a TaqMan probe that contains the sequence mostly specific to the targeted short RNA).

In one embodiment of this invention the bound complex is formed with 8 or fewer base pairs, such as 6 or fewer, between the target short RNA and the ligation agent. In another embodiment of this invention the bound complex is formed with 4 or fewer base pairs between the short RNA and the ligation agent. In another embodiment of this invention the bound complex is formed with 1, 2 or 3 or base pairs between the short RNA and the ligation agent.

Thus, the present invention provides methods of generating a ligated molecule to aid in the characterization and detection of target short RNAs. Short RNAs are small in size and are thus difficult to detect using standard detection methods. In some embodiments, the methods of the present invention comprise adding a ligation agent to a target short RNA to generate a bound complex. Such bound complexes can then be ligated with a template-dependent ligation enzyme to form a ligated molecule; the resulting extended molecule can then be detected by detection methods known in the art using all of the specific sequence comprised in the short RNA.

In some embodiments, the ligation agents and/or the target short RNA used to form bound complexes comprise one or more nucleotide analogs. For example, in some embodiments, 2′-O-methyl nucleotides are utilized. The present invention is not limited to a particular analog, mimetic or mechanism. Indeed, an understanding of the mechanism is not necessary to practice the present invention. Nonetheless, it is contemplated that the presence of 2′-O-methyl bases increases the stability of the hybridized bound complex and aids in further ligation and detection protocols.

Thus the present invention provides methods of detecting short RNAs. The present invention is not limited to a particular detection assay. Any suitable method may be utilized including, but not limited to, those disclosed herein. In some preferred embodiments of the present invention, short RNA detection methods are quantitative. The present invention is not limited to a particular mechanism. Indeed, an understanding of the mechanism is not necessary to practice the present invention. Nonetheless, it is contemplated that levels of a particular short RNA in the body are associated with a level of gene expression from their cognate genes. The present invention thus provides methods of correlated short RNAs with gene expression of particular genes (e.g., genes involved in disease states or metabolism). For example, in some embodiments, the methods of the present invention are utilized to determine the presence of abnormal (e.g., high or low) levels of a particular short RNA or to determine the effect of an intervention (e.g., drug) on short RNA expression. In other embodiments, heterologous short RNAs (e.g., from expression vectors, transgenic constructs, transfection, etc.) are detected to characterize the efficiency of short RNA expression systems. In some embodiments, the present invention provides methods of detecting a particular short RNA. In other embodiments, the methods of the present invention are used to distinguish between variants (e.g., polymorphisms or mutations) in a particular short RNA.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in molecular biology, genetics, or related fields are intended to be within the scope of the following claims.

### EXAMPLE 1

As a model to demonstrate the ability to detect short RNA, a detection of a 21 nucleotide long synthetic RNA siC12as as shown in Table 1 was performed.

TABLE 1
Oligonucleotides used in Examples

| oligonucleotide | sequence, 5′->3′ |
|---|---|
| Target short RNA* | ATTGGAGTGAGTTTAAGCTTT |
| siC12as | |
| Ligation primer | CGACTCATGGTCATAGCTGTTAGTCGAAAGCTTAAAC |
| LIGsiC12as | |
| REVsiP' | CTTCGAATTCGACTAACAGCTATGACCA |
| FORsiP' | CTTTCTAGATTGGAGTGAGT |
| TaqMan ™ probe | FAM-TGAGTCGAAAGCTTAAACT-Tamra |
| Alternative Ligation primer | CGACTCATGGTCATAGCTGTTAGTCGAGCTTAAAC |
| LIGshC12as | |

*RNA oligo; the sequence is shown as DNA

Ligation reaction. 2.4×10⁹ and 2.4×10⁷ copies of the siC12as RNA were mixed with 7 pmols of ligation primer LlGsiC12as (Table 1) in 15 μl of water. The mixture was heated at 95° C. for 1 min and placed on ice. To the mixture were added: 2 μl of 10×DNA Ligase buffer; 1 μl DNA ligase (from NEB) and 2 μl water. The mixture was incubated at 20° C. for 2 hours; then heat inactivated at 65° C. for 10 min.

Reverse transcription. 5 μl of ligated mixtures above corresponding to 0.6×10⁹ and 0.6×10⁷ copies of input short RNAs were mixed with 45 μl water, 10 μl 10×RT buffer, 4 μl 25 mM each of dNTPs, 10 μl of 20 μM primer REVsiP′ (Table 1), and 5 ul of reverse transcriptase (all supplied in Applied Biosystems High-Capacity cDNA Archive Kit). The mixtures were incubated 25° C. for 10 minutes and continued for additional 120 min at 37° C.

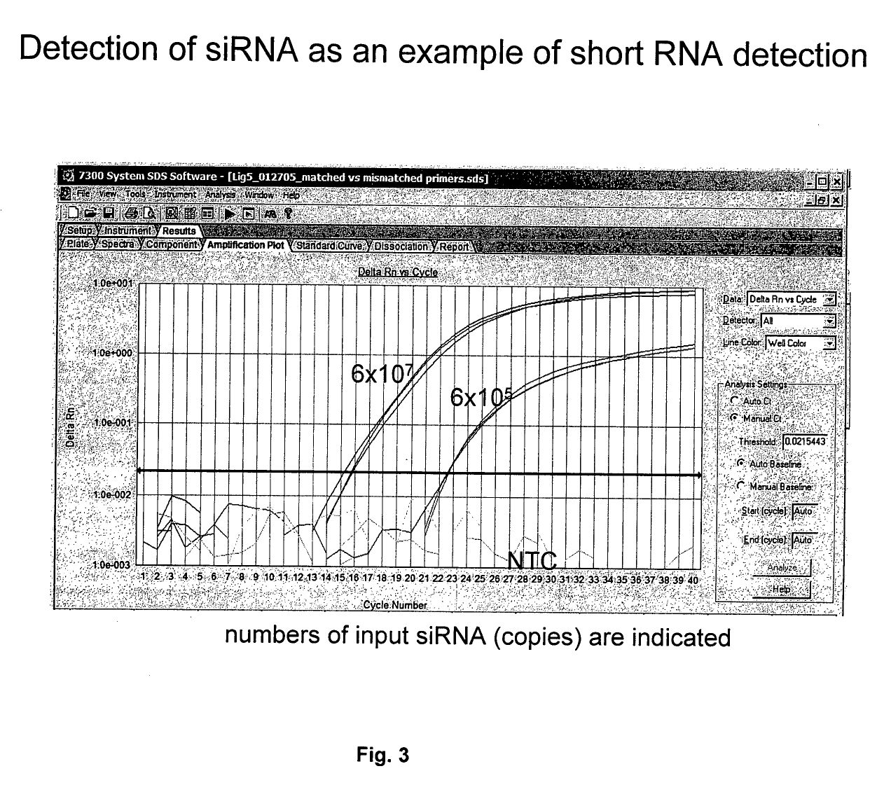

TaqMan™ detection. 10 μl of the templates from above (corresponding to 6×10⁷ and 6×10⁵ copies of input RNA) were mixed with 25 μl of Universal PCR Master mix (Applied Biosystems), 4 μl each of 11 μM forward primer FORsiP′ and reverse primer REVsiP′ (Table 1), 2 μl of 6 μM TaqMan probe (Table 1) and 5 μl water. Three repeats of each sample were tested. 40 cycles TaqMan™ program was performed. Results are shown on FIG. 3 and indicate that 21 nucleotide shotr RNA can be efficiently detected. No template control (NTC) samples indicated that the amplification and detection is target specific.

### EXAMPLE 2

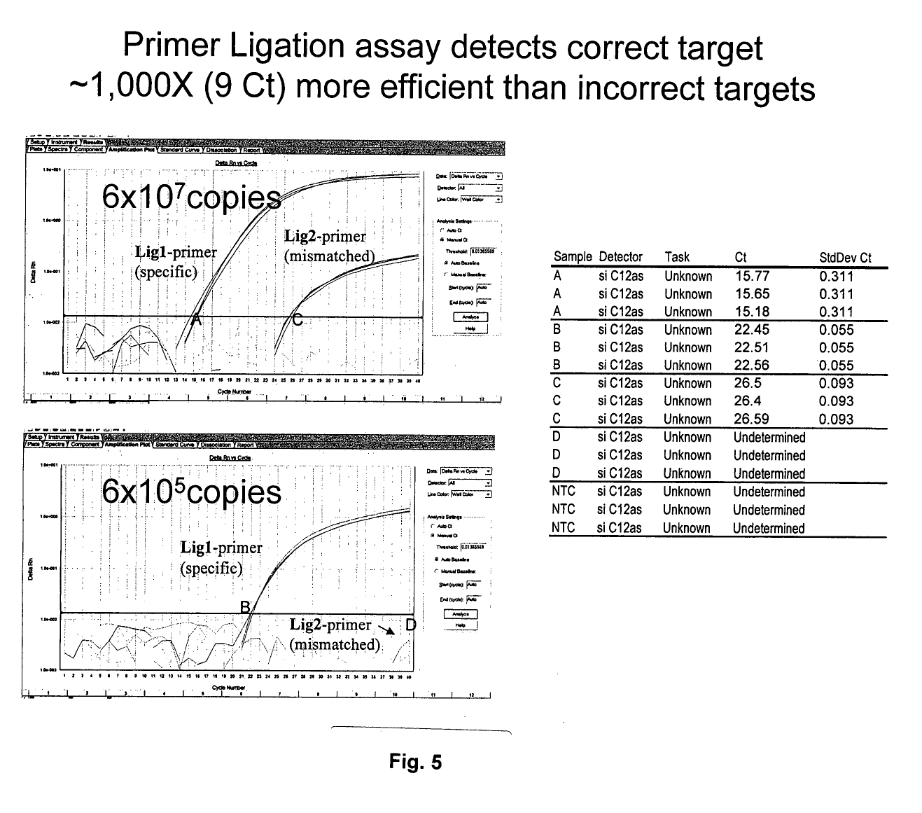

FIG. 4 schematically explains additional specificity added by the Ligation assay. Ligation primer can ligate to a target only if its structure precisely matches the target's 3′ end. Gaps or overlaps formed during formation of the bound complex prevent template dependent ligation using template-dependent ligase. To test this, the experiment similar to described in Example 1 was performed. Target specific ligation primer LlGsiC12as was compared to a similar primer LlGshC12as, that is equal to the LlGsiC12as except that it forms an overlap structure as in FIG. 4, right panel. The results of detection are shown on FIG. 5 and demonstrate that the target is recognized approximately 1,000× (˜9 Ct's difference) more efficient with a specific ligation primer.

What is claimed is:

1. A method of detecting a target short RNA, comprising:
hybridizing at least one ligation agent to the target short RNA;
ligating the at least one ligation agent and the target short RNA with a template-dependent ligase to form a ligated molecule; and
detecting the ligated molecule.

2. The method of claim 1, wherein the at least one ligation agent is a nucleic acid.

3. The method of claim 2, wherein the nucleic acid is a DNA oligonucleotide.

4. The method of claim 1, wherein a 3′ end of the ligation agent is not a substrate for a chain extension enzyme.

5. The method of claim 4, wherein the 3′ end of the at least one ligation agent is blocked by a hydrogen, 3′phosphoglycolate, or 3′amine.

6. The method of claim 1, wherein the target short RNA has a length of 17-50 nucleotides.

7. The method of claim 1, wherein the target short RNA is an mRNA, siRNA, or processed shRNA.

8. The method of claim 1, wherein the at least one ligation agent comprises a portion of sequence that is not complementary to the target short RNA and a portion of sequence that is complementary to the target short RNA.

9. The method of claim 1, wherein the ligated molecule is detected by a quantitative nucleic acid amplification technique.

10. The method of claim 9, wherein the ligated molecule is detected by a real-time PCR assay.

11. The method of claim 9, wherein the ligated molecule is detected by a TaqMan™ assay.

12. The method of claim 1, wherein the ligated molecule is detected by a method selected from the group consisting of sequencing assays, polymerase chain reaction assays, hybridization assays, hybridization assays employing a probe complementary to a mutation, microarray assays, bead array assays, primer extension assays, enzyme mismatch cleavage assays, branched hybridization assays, NASBA assays, molecular beacon assays, cycling probe assays, ligase chain reaction assays, invasive cleavage structure assays, ARMS assays, and sandwich hybridization assays.
